(19) United States
(12) Patent Application Publication
(10) Pub. No.: US 2025/0268616 A1
(43) Pub. Date: Aug. 28, 2025

(54) LITHOTRIPSY DEVICE FOR BREAKING UP CALCULI WITH AN AXIALLY MOVABLE ACCELERATION TUBE, AND METHOD FOR ACCELERATING A PROJECTILE OF A LITHOTRIPSY DEVICE

(71) Applicant: KARL STORZ SE & Co. KG, Tuttlingen (DE)

(72) Inventors: Florian HUBER, Tuttlingen (DE); Simon LEHMANN, Tuttlingen (DE)

(21) Appl. No.: 18/856,969

(22) PCT Filed: Apr. 3, 2023
(86) PCT No.: PCT/EP2023/058667

§ 371 Date: Oct. 15, 2024

(30) Foreign Application Priority Data
Apr. 13, 2022 (DE) 10 2022 109 140.6

(51) Int. Cl. A61B 17/22; A61B 17/00

(52) U.S. Cl. CPC A61B 17/22012; A61B 2017/00544; A61B 2017/00862; A61B 2017/22025

(57) ABSTRACT

A lithotripsy device breaks up calculi and includes: a carrier unit; a guide tube; an acceleration tube having an axial direction, a cavity, a proximal end, a distal end; a movable projectile; a proximal stop element and a distal stop element for the movable projectile. The acceleration tube includes a proximal distal opening for the inflow and/or outflow of a pressure medium into and/or out of the cavity to move the projectile back and forth between the proximal and distal stop elements, the acceleration tube being positioned so as to be movable in the axial direction internally at a proximal end portion by the proximal stop element and at a distal end portion by the distal stop element so that the acceleration tube can be displaced in a distal direction and in a proximal direction relative to the guide tube. A method accelerates a projectile of a lithotripsy device.

### CROSS REFERENCE TO RELATED APPLICATIONS

This application is a United States National Phase Application of International Application PCT/EP2023/058667, filed Apr. 3, 2023, and claims the benefit of priority under 35 U.S.C. § 119 of German Application 10 2022 109 140.6, filed Apr. 13, 2022, the entire contents of which are incorporated herein by reference.

### TECHNICAL FIELD

The present invention relates to a lithotripsy device for breaking up calculi, wherein the lithotripsy device comprises a carrier unit, a guide tube, an acceleration tube with an axial direction, a cavity, a proximal end and a distal end, a movable projectile, and a proximal stop element and a distal stop element for the movable projectile, wherein the acceleration tube is at least partially surrounded by the guide tube and the acceleration tube has at least one proximal opening and at least one distal opening for the inflow and/or outflow of a pressure medium into and/or out of its cavity in order to move the projectile back and forth between the proximal stop element and the distal stop element. A drive device for supplying and/or discharging the pressure medium, and a probe can be assigned to the lithotripsy device, wherein the probe can be connected directly or indirectly to the carrier unit at its proximal end, and can be excited to vibration by a mechanical impact of the projectile on the distal stop element. Furthermore, the invention relates to a method for accelerating a projectile of a lithotripsy device.

### BACKGROUND

Lithotripsy is a well-known method for fragmenting calculi, which form as so-called concretions in body organs, for example in the bladder or kidneys, due to the condensation and/or crystallization of salts and proteins. If the calculi are too large for natural passage and cause discomfort, they must be comminuted using a lithotripter so that the comminuted stones can be removed by natural excretion and/or by means of a suction/rinsing pump. The calculi to be comminuted are often inhomogeneous, with different constituents and/or solidities.

Pneumatic lithotripters are based on the percussion hammer principle, in which a projectile is accelerated within a usually permanently installed acceleration tube, and the kinetic energy of the projectile is transferred via an elastic impact to the proximal end of a probe and/or sonotrode and onward to its distal end in order to fragmentize the calculus. Usually, the successive impacts of the projectile are controlled by timed pulses of compressed air. As a result, the tempo of the shock waves transmitted to the probe and/or sonotrode is directly dependent on the temporal sequence of the compressed air pulses successively applied. Consequently, in known lithotripters the impact cadence is limited due to the single-lumen acceleration tube and reversing compressed air propulsion of the projectile. In addition, a compressed air reservoir must be connected on the distal end to the interior of the acceleration tube via a connection and/or a switching valve in order to move the projectile back to the proximal stop after it has struck the distal end. In the simplest case, a passive air spring is used to repulse the projectile; in this case, the projectile moving in the distal direction displaces the air from the acceleration tube into a reservoir within which the pressure increases. After the acceleration pressure in the distal direction has been switched off, the pressure in the reservoir can be used to move the projectile back in the proximal direction. The disadvantage here is that the acceleration pressure built up in the distal direction damps the repulsion of the projectile in the proximal direction, and the energy that can be stored in the reservoir is limited, which means that the projectile accelerates backward more slowly. Accordingly, the air in the connecting tube to the lithotripter must also be moved backward with each pulse and escape to the outside via a resistance of a proximal switching valve, for example in the control unit. In addition to a pressure regulation, a complex operating device with a time-controlled changeover valve is thus required. In addition, the projectile usually does not automatically spring back at the proximal stop and thus at the reversal point but must be accelerated again from a standstill in the distal direction by means of compressed air. These boundary conditions usually limit the maximum percussion cadence to well below 15 Hz.

Furthermore, a known device usually requires a reversing lever to change the direction of movement of the projectile and thus to redirect the impact. Due to a loss of percussive impact caused by a reversing lever, the generation of a high distal velocity with a simultaneous high amplitude at the sonotrode and/or probe end is only possible to a limited extent.

DE 10 2020 117 713 A1 discloses a lithotripsy device having an ultrasound unit and a shock pulse unit, wherein the shock pulse unit has a guide tube with a guide channel and a constant acceleration path in which the projectile is movably mounted. The projectile is accelerated pneumatically by means of a drive device by the transmission of compressed air pulses to the guide channel, and, at the end of the acceleration path, strikes a transmission element mounted floatingly in a coupling unit, such that a shock pulse is transmitted from the projectile to the transmission element and from the transmission element onward to a sonotrode head.

DE 20 2010 001 176 U1 describes a medical pressure-wave device with a guide tube held in a housing in which an impactor is guided. The movement path of the impactor along the interior of the guide tube is limited by a proximal stop built into a proximal end cap and by an impact body on the opposite distal end. The impact body is attached within the distal end cap by means of O-rings. The impactor is driven pneumatically by means of a compressed gas supply device, which has a pneumatic compressor which supplies a compressed gas connection of the handpiece of the pressure wave device via an external pressure line and a switching valve, which compressed gas connection is connected to the guide tube via an opening. By opening the switching valve and applying supply pressure via the compressed gas connection to the guide tube, the impactor is accelerated in the direction of the impact body. Even before the impactor strikes the impact body, the pressure is reduced again by switching the switching valve back. A return movement of the impactor immediately after impact with the impact body is facilitated by a counterpressure chamber which is connected to the distal end of the guide tube. By means of the counterpressure generated by the counterpressure chamber, the impactor is moved back to the proximal stop. For a new triggering process and thus a movement of the impactor in the distal direction, the switching valve must be switched again.

DE 20 2010 007 860 U1 relates to a pressure-wave device with a pneumatic drive for generating a pressure wave, having a housing and a contact device for contacting a human or animal body. In this case, the contact device is mounted on the housing via a force sensor in such a way that a force transmission from the contact device to the housing takes place at least partially via the force sensor, so that a contact force can be measured by a user. The contact device is mounted in a shaft of the housing in such a way that a relative displacement between the contact device and the housing along the longitudinal axis of the housing is possible. A guide tube is held in the attachment device, wherein a relative displacement between the housing and the contact device with the guide tube proceeds under force applied by a proximal spring body. The relative displacement between the guide tube and the housing that occurs when the pressure-wave device is used provides a force value via the force sensor, which essentially corresponds to the contact force. An impactor is guided in the guide tube, which can be accelerated in the distal direction by a pressure pulse of a compressed gas, wherein the movement in the distal direction is limited by an impact body elastically mounted against the contact device.

Conventional lithotripters have the main disadvantage that they do not have an active reset and thus repulsion of the projectile, and instead have to rely on slow and inefficient air springs formed between the projectile and the inside of the acceleration tube. This limits the usable impact frequency and once a limit frequency is reached can lead to it no longer being possible to use the acceleration path in its entirety. If the changeover valve is located externally in a drive and/or supply device, a great deal of pressure is typically lost in an elongation of the supply hose for the compressed air. On the other hand, the use of a stiffer hose with lower pressure loss impairs the handling of the lithotripter. Installing the valve and/or the entire drive and supply unit in the handpiece of the lithotripter makes sterilization more difficult and increases the weight.

### SUMMARY

It is an object of the invention to improve upon the prior art.

The object is achieved by a lithotripsy device for breaking up calculi, wherein the lithotripsy device comprises a carrier unit, a guide tube, an acceleration tube with an axial direction, a cavity, a proximal end and a distal end, a movable projectile, and a proximal stop element and a distal stop element for the movable projectile, wherein the acceleration tube is at least partially surrounded by the guide tube and the acceleration tube has at least one proximal opening and at least one distal opening for the inflow and/or outflow of a pressure medium into and/or out of its cavity in order to move the projectile back and forth between the proximal stop element and the distal stop element. A drive device for supplying and/or discharging the pressure medium, and a probe can be assigned to the lithotripsy device, wherein the probe can be connected directly or indirectly to the carrier unit at its proximal end, and can be excited to vibration by a mechanical impact of the projectile on the distal stop element, wherein the acceleration tube is arranged so as to be movable internally in the axial direction by means of the proximal stop element at its proximal end portion and by means of the distal stop element at its distal end portion, so that the acceleration tube is displaceable in a distal direction and in a proximal direction relative to the guide tube.

In this way, a lithotripsy device is provided, having an axially movable acceleration tube in which friction losses are minimized due to a very short fit of the proximal end portion with the proximal stop element and the distal end portion with the distal stop element, such that the complete acceleration path and thus the length of the acceleration tube in its cavity can be utilized to accelerate the projectile.

Because the acceleration tube has at least one proximal opening and at least one distal opening for the inflow and/or outflow of a pressure medium into and/or out of its cavity, and the acceleration tube is arranged to be axially movable relative to the surrounding guide tube, the position of the at least one proximal opening and of the at least one distal opening can also be directly adjusted relative to the guide tube by moving the acceleration tube in the axial direction. Due to the axial mobility of the acceleration tube and thus its targeted displacement in the axial direction, the at least one proximal opening and the at least one distal opening are correspondingly displaced relative to the guide tube, and can be used directly to control the supply and/or discharge of the pressure medium and thus to accelerate the projectile in the distal direction or in the proximal direction. Consequently, a self-controlling pneumatic drive is provided by means of the axially movable acceleration tube. This allows the projectile to be accelerated over a multiple of its length along the acceleration path in order to transfer to the probe the momentum of the projectile upon impact with the distal stop element for pneumatic stone fragmentation.

In this way, after the pressure medium feed has been started once, a self-controlling process takes place in which the projectile is continuously moved back and forth by the constant pressure of the pressure medium, wherein the switching of the direction of movement is predetermined by the axial position of the acceleration tube and of the at least one proximal opening and/or of the at least one distal opening. The uniform, constant pressure medium flow through the at least one proximal opening or the at least one distal opening of the acceleration tube and the substantially complete utilization of the acceleration path make possible a higher impact cadence, in particular with a frequency of >15 Hz, preferably >30 Hz, than is the case with known lithotripters. In addition, it is ensured that the entire acceleration path is used and the impact effect does not diminish with increasing frequency, which means that a greater stone removal performance can be achieved than with known lithotripters. Accordingly, for the same projectile velocity, the lithotripsy device according to the invention can also be operated with a lower pressure. In addition, compared to known lithotripters the lithotripsy device has a smaller installation space and thus a reduced instrument weight, since the distal pressure reservoir with and/or without switching valve and with connection to the acceleration path is omitted.

An essential concept of the invention pertains to the fact that, contrary to the conventional view that an acceleration tube must be fixedly installed within a lithotripsy device, the acceleration tube is designed to be movable in the axial direction, and a valve switch for moving the projectile back and forth is realized by moving the acceleration tube in the axial direction or in the proximal direction. By integrating the valve switch and thus the redirection of the direction of movement of the projectile by means of the axially movable acceleration tube directly within the lithotripsy device, a complex external feed and discharge, and also regulation, of the pressure medium, a time controller for the external switching valves, and a distal pressure reservoir are not required. In addition, the frequency of the mechanical impacts of the projectile on the probe is not predetermined by external, clocked pressure pulses, but is specifically adjustable via the pressure medium flow according to the axial position of the acceleration tube and thus of the at least one proximal opening and of the at least one distal opening, and thus of their presence in an overpressure range (alternatively, a negative pressure range) or in the ambient pressure range, and the direction of movement of the projectile can be specified.

The following terminology is explained:

A “lithotripsy device” (also known as a “lithotripter”) is in particular a device for comminuting calculi by means of impacts, impact waves, and/or deformation waves. A lithotripsy device is understood to mean in particular various components, structural and/or functional elements of a lithotripter. The lithotripsy device can completely or partially form a lithotripter. A lithotripsy device can in particular be an intracorporeal or extracorporeal lithotripsy device. In the case of an intracorporeal lithotripsy device, this can also have a rinsing/suction pump. The lithotripsy device can be designed as a hand-held device and/or have an endoscope or be inserted into an endoscope. The lithotripsy device is in particular autoclavable and comprises, for example, instrument steel and/or plastics. The lithotripsy device can have further components, such as a control and/or supply unit, or these are assigned to the lithotripsy device. A lithotripsy device is in particular a pneumatic lithotripsy device. In principle, the lithotripsy device can also have a combined excitation with a repetitive impact excitation by means of the projectile, and a constant vibration excitation, for example by means of an ultrasound generator. For this purpose, the lithotripsy device has in particular a counter-bearing, a horn and at least one piezo element as a vibration exciter between the counter-bearing and the horn, wherein the horn can in particular be connected to the probe and the at least one piezo element can be electrically connected to an assignable ultrasound generator, such that a substantially constant ultrasound energy can be supplied to the probe by means of the piezo element. In the case of combined excitation, the probe is preferably designed as a solid rod sonotrode. Furthermore, the lithotripsy device and/or the carrier unit comprises an operating unit for starting, stopping and/or individually triggering a movement of the projectile. An operating unit can be, for example, a lever, a push button and/or a rotary knob. By operating the switching function on the lithotripsy device itself, the otherwise usual foot switch for triggering a shock wave of the projectile can be dispensed with, thus reducing the costs of the lithotripsy device and improving clarity in the operating room.

“Calculi” (also known as “concretions”) are understood to mean in particular all stones in a human or animal body that are formed from salts and proteins through crystallization and/or condensation. Calculi can be, for example, gallstones, urinary stones, kidney stones and/or salivary stones.

A “carrier unit” is in particular a hand and/or holding part of the lithotripsy device. In particular, the carrier unit can be a handle for manual and/or automated operation and/or connection of the lithotripsy device. The carrier unit can also be arranged, connected and/or automatically guided at a distal end of a robot arm. In particular, the carrier unit has a housing.

A “guide tube” is in particular an elongated hollow body whose length is greater than its diameter. The guide tube has in its interior in particular a cavity in which the acceleration tube is at least partially arranged. In particular, the guide tube can have the same length as the acceleration tube or a shorter length than the acceleration tube. With a shorter length of the guide tube, a proximal end cap can be arranged at its proximal end and a distal end cap can be arranged at its distal end, wherein the acceleration tube can be arranged further in the axial direction in a cavity of the proximal and distal end caps. At least one proximal inlet air channel and at least one proximal outlet air channel and/or at least one distal inlet air channel and one proximal outlet air channel can be created by means of the guide tube, the proximal end cap and/or the distal end cap; through these, the pressure medium flows into or out of the at least one proximal opening and the at least one distal opening of the acceleration tube. Inlet air and outlet air channels in the guide tube can be designed as cavities so that the pressure medium can flow into and/or out of the acceleration tube from either end. The cavity of the guide tube itself is preferably under ambient pressure conditions. Instead of a tube, a guide tube can also be a hollow cylinder, wherein the two completely closed and/or partially closed end faces directly or indirectly (for example, by means of a spring arranged between them) form a proximal and a distal stop end for the proximal stop element and distal stop element, which are also axially movable. The guide tube can also be designed as two axially oriented guide elements, which are each arranged at least around the proximal end and the distal end of the acceleration tube. In principle, the cross-section of the guide tube does not have to be circular, but can have any shape, such as oval, triangular, square or polygonal. In particular, the guide tube is fixed inside the carrier unit of the lithotripsy device and is therefore not movable.

A “stop element” is in particular an element or component as the intended end point of the movement of the projectile along the acceleration path, at which the accelerated projectile impacts, is braked by, is sent back by a spring and/or is repulsed and/or moved in the opposite direction. In this way, the stop element absorbs at least part of the kinetic energy of the projectile. The distal stop element absorbs in particular the impact and/or shock of the projectile and transmits it directly or indirectly to the probe. A proximal stop element is arranged in particular at and/or in the proximal end of the acceleration tube and/or within the cavity in a region of the proximal portion of the acceleration tube. Accordingly, a distal stop element is arranged in particular on and/or in the distal end of the acceleration tube and/or within the cavity in a region of the distal end portion of the acceleration tube. In particular, the distal stop element is directly or indirectly connected to the proximal end of the probe. The proximal stop element and the distal stop element have a substantially cylindrical shape with their longitudinal center axis oriented parallel to the longitudinal center axis of the acceleration tube. The proximal stop element and the distal stop element are movable in particular in the axial direction, distal direction and/or proximal direction. The proximal stop element and/or the distal stop element in particular comprise a hard material, such as stainless steel or hardened steel and/or a hardening layer, such as a carbon layer (diamond-like carbon). Preferably, the proximal stop element and/or the distal stop element is/are harder than the projectile or vice versa. Preferably, one of the two impact partners is softer than the other. The proximal stop element and the distal stop element can each form a pneumatic spring inside the cavity of the acceleration tube with the surrounding acceleration tube. Preferably, however, there is no pneumatic spring, but instead the proximal stop element and the distal stop element each abut a separate spring element. In particular, the distal stop element (also called “billiard projectile”) has a slightly higher mass than the projectile. The mass ratio of projectile to billiard projectile is in particular in a range of 0.6 to 1.4, preferably close to 1:1 and optimally at 1:1.2, in order to optimally achieve a 100% energy and momentum transfer for an elastic collision in the latter case. In particular, the mass ratio of the proximal stop element to the projectile should be in a region around 1:1. Due to this mass ratio, in the event of an impact on a spring-loaded, proximal stop element, the projectile transfers the entire impulse to the proximal stop element and, via a first spring element, a force is further transferred to the acceleration tube, which displaces it in the proximal direction up to a proximal stop of the acceleration tube, and a starting position for renewed acceleration of the projectile in the distal direction is present.

“Distal end” and “distal” are understood to mean an arrangement and/or a corresponding end or portion that is close to the body and therefore remote from the user. Accordingly, “proximal end” and “proximal” are understood to mean an arrangement and a corresponding end or portion close to the user and thus remote from the body. Accordingly, a “distal direction” is understood to mean the direction oriented toward the distal end of the acceleration tube and/or of the lithotripsy device. The distal direction is in particular the direction of movement of the projectile towards the probe. A “proximal direction” is understood to mean in particular the direction towards the proximal end of the acceleration tube and/or of the lithotripsy device. In this way, the proximal direction is the direction of backward or returning movement of the projectile.

An “acceleration path” is in particular a portion of a longitudinal dimension of the cavity of the acceleration tube, which is defined by a distal stop surface of the proximal stop element and by a proximal stop surface of the distal stop element. In particular, the maximum acceleration path of the projectile corresponds to the maximum longitudinal dimension of the cavity of the acceleration tube minus the projectile length when the proximal stop element is flush with the proximal end of the acceleration tube and the distal stop element is flush with the distal end of the acceleration tube. The longitudinal dimension of the cavity can, for example, be 150 mm.

A “projectile” is in particular a body which is freely movable along the acceleration path in the axial direction within the cavity of the acceleration tube. In particular, the projectile can be moved back and forth between the proximal stop element and the distal stop element within the cavity of the acceleration tube arranged therebetween. In principle, the projectile can have any form. For example, the projectile may have the form of a bolt or a ball. The projectile may have a slightly smaller diameter at its proximal end than in the central region. In this way, the projectile can have a chamfer at its proximal end portion, which, for example, widens conically from the proximal end to the central region of the projectile. Likewise, the projectile may have a chamfer at its distal end portion and thus narrow from a central region to the distal end. Such a chamfer on both ends of the projectile particularly improves the movement at a starting point and/or reversal point of the projectile. Furthermore, the projectile can have structuring on its surface, such as grooves. This minimizes contact with the inner surface of the acceleration tube. The projectile in particular has hard steel and/or magnetic properties. In order to ensure free mobility, the projectile has a slightly smaller outer diameter than the diameter of the cavity of the acceleration tube. For example, the projectile may have an outer diameter of 8 mm, preferably 6 mm.

The projectile can be moved back and forth in particular between the proximal stop element and the distal stop element and thus along the acceleration path continuously by means of the pressure medium of the drive device. Preferably, the projectile is continuously moved intermittently and/or oscillatingly back and forth between the proximal stop element and the distal stop element.

The “acceleration tube” is in particular an elongated hollow body whose length is greater than its diameter. In particular, the acceleration tube has a cavity inside it that runs axially and within which the projectile can move. The acceleration tube is in particular tubular with an open proximal end and an open distal end, wherein the proximal stop element can be arranged and moved at least partially within the open proximal end and the distal stop element can be arranged and moved at least partially within the open distal end. The acceleration tube can also be designed as a hollow cylinder, with at least one opening arranged in each of the end faces. In particular, the acceleration tube has a smaller diameter than the guide tube. The acceleration tube has at least one proximal opening and at least one distal opening for the passage of the pressure medium. The acceleration tube can in particular be arranged fixed against rotation in the cavity of the guide tube and/or the end caps at the respective ends, so that an axial movement of the acceleration tube is possible but not a rotation of the acceleration tube, such that the proximal opening and/or the distal opening of the acceleration tube with the corresponding inlet air channel or outlet air channel in the guide tube and/or the two terminal end caps can be brought into a passage position for the pressure medium. If the inlet air and outlet air channels in the guide tube are designed as cavities, in particular as circumferential cavities around the outer surface of the acceleration tube, an anti-rotation arrangement is not required.

The acceleration tube and its at least one proximal opening and at least one distal opening are designed in particular such that in the case of, for example, a proximal stop of the proximal stop element and the proximal end of the acceleration tube, the proximal opening of the acceleration tube is arranged to create a passage with the proximal inlet air channel, and pressure medium can flow through the inlet air channel and the proximal opening into the cavity for accelerating the projectile in the distal direction. Likewise, when the distal stop element and the distal end of the acceleration tube strike each other distally, the distal opening is connected to the distal inlet air channel and the projectile in the cavity is moved back in the proximal direction by the inflowing pressure medium. The projectile can rebound accordingly at the respective stop elements.

A “longitudinal center axis” is in particular the axis of the acceleration tube and/or of the lithotripsy device which corresponds to the direction of the greatest dimension of the acceleration tube and/or of the lithotripsy device. The longitudinal center axis thus runs along the axial direction.

“Radial” is understood to mean running in the direction of a radius. In this way, the radial direction runs from the longitudinal center axis outwards.

A proximal and a distal “opening” are each a breakthrough through a wall of the acceleration tube. The proximal opening and the distal opening are in particular formed continuously through the outer peripheral surface of the acceleration tube. The proximal opening and the distal opening of the acceleration tube can each be a hole. In particular, these openings have a relatively large diameter, so that essentially no pressure loss occurs. For example, the openings of the acceleration tube can have a diameter in a range of 2 to 3 mm with an acceleration tube diameter of 6 mm. The openings of the acceleration tube may have a chamfer inside toward the cavity thereof to prevent wear and/or chip formation on the projectile.

In principle, a “drive device” can be any type of device which, by feeding and/or discharging a pressure medium, exerts a force on the projectile and thus causes the projectile to move. The drive device causes in particular a continuous and uniform inflow of the pressure medium through the proximal or distal openings of the acceleration tube, for example pneumatically by means of compressed air, and an acceleration of the projectile within the cavity of the acceleration tube.

A “pressure medium” is in particular a fluid. A pressure medium can be a gas, such as compressed air. The pressure medium can, for example, be taken from a building pressure supply line and/or generated by a compressor. The pressure medium is in particular continuously supplied to and/or removed from the lithotripsy device and/or circulated. In particular, the pressure medium has a pressure in a range of 0 to 10 bar. Due to the continuous supply and removal of the pressure medium free from alternating loads, a pressure of >10 bar can also be used.

A “probe” is in particular an elongated component which is, for example, rod-, tube- and/or hose-shaped. The probe can also be a hollow probe which has a cavity inside it, at least partially or completely, in the longitudinal direction. The hollow probe has at its distal end in particular a distal opening which is connected to the inner cavity. The probe can itself be set into vibration, resonance vibration and/or deformation vibration, in particular by the action and/or introduction of mechanical vibrations. A probe may also be a sonotrode. In particular, the probe is designed in one piece. In particular, the probe has a diameter in the range from 0.5 mm to 4.5 mm, in particular from 0.8 mm to 3.8 mm. The probe is made of steel, titanium, aluminum and/or carbon. A probe may in particular be a single-use probe or a multiple-use probe.

In a pneumatic lithotripsy device, a specifically shaped deformation wave is produced by means of impact energy when a projectile strikes a distal stop element, in particular the probe. In particular, the deformation wave causes a translational movement of the probe, which, due to the deflection, results in improved stone comminution. In addition to the mechanical impact, the probe can additionally be excited to vibration, in particular longitudinal vibration, in particular by means of a vibration excitation device, for example by means of an ultrasonic vibration exciter. In this way, the probe is designed in particular as a waveguide for the vibration waves generated by a vibration excitation device and/or for the shock waves and/or deformation waves of the projectile.

In particular, the proximal end of the probe can directly or indirectly abut the distal stop element. Preferably, the probe is fitted on the proximal end into a threaded/retaining nipple that is thicker than its diameter. A corresponding nipple can also be a head piece. Preferably, the head piece of the probe is mounted so that it can move. In particular, the probe is shaped in such a way that it optimally introduces the vibration waves, deformation waves, impact waves and/or the ultrasonic vibrations at its distal end into the body, the body region to be treated and/or directly onto the calculus to be fragmented.

In a further embodiment of the lithotripsy device, there is, for a proximal stop of the axially movable acceleration tube, a first valve opening position for the pressure medium to flow through the at least one proximal opening into and/or out of the cavity of the acceleration tube, in order to move the projectile towards the distal stop element or to move the projectile back to the proximal stop element, and there is, for a distal stop of the axially movable acceleration tube, a second valve opening position for the pressure medium to flow through the at least one distal opening into and/or out of the cavity of the acceleration tube, in order to move the projectile back to the proximal stop element or to move the projectile towards the distal stop element.

In this way, depending on the position of the movable acceleration tube in the axial direction, a defined first valve opening position or a defined second valve opening position is made possible, such that the acceleration tube is moved further axially in the direction of movement of the projectile by impact of the projectile on the distal stop element or the proximal stop element, such that the flow direction of the pressure medium is switched and thus the direction of movement of the projectile and also of the axially movable acceleration tube is changed.

In principle, it should be noted that the pressure medium can flow into or out of the cavity of the acceleration tube through the at least one proximal opening or the at least one distal opening. If, for example, an inflow of the pressure medium through at least one proximal opening and thus an overpressure operation is realized at a proximal stop of the proximal end of the acceleration tube, the projectile will be accelerated towards the distal stop element. For negative pressure operation and thus an outflow of the pressure medium from the cavity, a corresponding negative pressure must be applied to the distal opening of the acceleration tube so that the projectile moves from the proximal end of the acceleration tube to the distal stop element. In this way, depending on their axial position, the proximal opening and the distal opening are in the overpressure range (alternatively negative pressure range) or in the ambient pressure range, thereby controlling the direction of movement of the projectile. The switching of the direction of movement is induced by the projectile striking the proximal stop element or the distal stop element, such that a simultaneous impact on the probe occurs at the distal end. This reverses the compressed air supply or exhaust at the acceleration tube to passively and/or actively move the projectile back. It is particularly advantageous that for supplying the pressure medium to the lithotripsy device only a single hose is required for supplying the inlet air (or to apply a negative pressure), and the energy for switching is taken from the projectile in the acceleration direction while it is still being accelerated by the inflowing (or outflowing) pressure medium. Depending on the movement of the acceleration tube in the distal or proximal direction according to the axial position, at least one proximal opening is open in the first valve opening position and thus can be flowed through by pressure medium and is closed in the second valve opening position to allow the pressure medium to flow in. For the distal opening, the valve opening positions behave correspondingly in reverse.

In order to realize a uniform, spatially distributed flow of the pressure medium, the acceleration tube has a second proximal opening and/or further proximal openings and a second distal opening and/or further distal openings.

The flow resistance can also be reduced by having multiple proximal and/or distal openings.

The “second proximal opening” or “the further proximal openings” and the “second distal opening” or “the further distal openings” are, in their respective design and function, a proximal opening or a distal opening as defined above. However, these additional proximal or distal openings may be located at a different position on the acceleration tube.

In a further embodiment of the lithotripsy device, the at least one proximal opening or openings and the at least one distal opening or openings are arranged in a lateral surface of the acceleration tube, axially symmetrical with respect to a longitudinal center axis of the acceleration tube, and/or all around the circumference.

By designing the respective proximal openings and the respective distal openings passing through the outer surface of the acceleration tube, thus giving a radial orientation transverse to the axial direction, the outflowing pressure medium is discharged laterally from the acceleration tube and/or the lithotripsy device. As a result, the venting direction is perpendicular to the distal direction, such that the pressure medium emerging from the cavity of the acceleration tube is preferably already present directly in an ambient pressure range before passing through an outlet air channel, and is released from there into the environment of the lithotripsy device. This prevents excess pressure of the pressure medium being directed distally towards the probe, thus removing the risk that a patient is exposed to undesirable excess pressure in the event of a malfunction.

Furthermore, the axially symmetrical arrangement of the respective proximal and/or distal openings or of their circumferential arrangement ensures a uniform inflow into and/or outflow from the cavity of the acceleration tube, such that the mobility of the acceleration tube in the axial direction is not influenced. In principle, it should be emphasized that the plurality of proximal and/or distal openings preferably have the same cross-sectional opening. However, they can also have cross-sectional openings of different sizes.

In order to arrange the proximal stop element and/or the distal stop element at least partially in the cavity of the acceleration tube and movably, the proximal stop element has a first cylindrical portion and the distal stop element has a second cylindrical portion, wherein the proximal end portion of the acceleration tube is arranged around the first cylindrical portion and the distal end portion of the acceleration tube is arranged around the second cylindrical portion so as to be movable in the axial direction.

In a further embodiment, the proximal stop element has a first end portion as a stop at the proximal end of the acceleration tube and the distal stop element has a second end portion as a stop at the distal end of the acceleration tube.

In addition to the formation of each of the defined stops, the respective end portions of the proximal stop element and the distal stop element also safely close off the pressurized area of the cavity of the acceleration tube. This, like the discharge of the exhaust air in the radial direction, prevents an undesirable overpressure in the distal direction toward the probe and thus toward a patient. The end portions also prevent the projectile from exiting the acceleration tube in the axial direction.

A “terminal portion” is in particular a region of the proximal and distal stop element which has a larger cross-section than the cross-section of the outer diameter of the acceleration tube, so that the proximal end and the distal end of the acceleration tube abut the radially outwardly projecting terminal portion on the proximal end and distal end, respectively, in the axial direction. In order to damp the respective stop, a damping element, such as an O-ring, can be arranged between the respective end of the acceleration tube and the end portion.

In order to actively move the projectile that has struck the stop element back in the opposite direction in addition to a passive reset, a first spring element for repulsing the projectile is arranged on the proximal end of the proximal stop element and/or the first end element.

In this way, an active proximal reversal mechanism is provided in which the kinetic energy of the projectile is transferred to the proximal stop element, this stop element compresses the spring element, which exerts a force on the acceleration tube and thus displaces it in the proximal direction. The remaining force can then be converted by the spring element back into kinetic energy of the proximal stop element and transferred by impact from this stop element to the projectile, thereby recovering part of the energy that was supplied to the projectile during the movement from the distal end of the acceleration tube to the proximal end of the acceleration tube. Optimally, the kinetic energy of the impacting projectile is largely stored in the spring element by means of the proximal first spring element and used for the axial movement of the acceleration tube and the repulsion of the projectile. In this way, the spring element promotes the rebound of the projectile and thus the reversal movement. In principle, it should be emphasized that at sufficient speed the projectile is also passively repelled and/or moved without a spring element on the proximal or distal stop element. However, in the case of this passive reset, a short dead time may occur at this reversal point. In order to reliably overcome this reversal point, the reversal of movement is actively initiated and accelerated by a first and/or second spring element of the distal stop element and/or the proximal stop element and consequently a rapid switching between the valve opening positions is achieved.

A “spring element” (also called a spring) is in particular any element and/or component which can be sufficiently elastically deformed to overcome a short-term counterpressure at the reversal point of the reversal of movement of the projectile at the distal stop element or proximal stop element. A spring element can, for example, be a coil spring and thus a wire wound in a spiral shape with sufficient energy storage capacity. The respective spring elements convert in particular the kinetic energy of the projectile, which is initially transferred to the proximal or distal stop element, into a tension energy, which is used for an axial movement of the acceleration tube and the active return of the projectile. In particular, the spring has a larger diameter than the projectile and/or a similar or larger diameter than the acceleration tube. For example, the spring element can have a diameter in a range of 5.00 mm to 9.00 mm and/or a wire thickness in a range of 0.50 mm to 1.25 mm. For example, a length of the spring element can be in a range of 5.00 mm to 10.00 mm in a relaxed state and in a range of 1.00 mm to 2.00 mm in a compressed state.

In a further embodiment, the distal stop element has an impact pin distally in relation to the second cylindrical portion and/or to the second end portion for transmitting an impact of the projectile to the probe.

In this way, the impact pin efficiently transmits the shock to the probe. In this case, it is particularly advantageous that the design of the impact pin, in particular its length and diameter, can be realized independently of the design of the remaining distal stop element. In this way, the diameter of the impact pin can be individually adapted to the diameter of the probe head on which the impact pin strikes. This enables efficient acceleration and active recoil of a projectile over a multiple of its own length in order to transfer its momentum to a probe for stone fragmentation.

An “impact pin” is in particular a distal portion of the distal stop element for transmitting a shock to the probe. The impact pin has in particular a pin-shaped and/or cylindrical shape. With the distal end face and/or circular surface, the impact pin strikes the proximal end of the probe, in particular directly or indirectly. The end face is in particular a smooth surface.

In order to also effect an active, axial displacement of the acceleration tube and optionally an active repulsion of the projectile at the distal stop of the projectile, a second spring element is arranged distally in relation to the second end portion and/or around the impact pin of the distal stop element.

It is particularly advantageous if the spring element, for example designed as a spiral spring, surrounds the outside of the impact pin. This allows the impact pin to be moved distally beyond the distal end of the spring and efficiently transmit a shock to the probe. This design of the distal stop element simultaneously enables efficient energy and shock transmission. When the projectile strikes the billiard projectile as a distal stop element, the momentum and kinetic energy of the projectile is transferred to the billiard projectile. The billiard projectile compresses the second spring element, which rests proximally on the second end element of the billiard projectile, and thus transfers a force to the acceleration tube, causing it to be displaced in the distal direction. The billiard projectile and the spring element are designed in such a way that the spring element is compressed just enough to move the acceleration tube sufficiently quickly. By choosing an appropriate spring hardness, it is possible to adjust how much energy is transferred to the acceleration tube. The billiard projectile still has a residual velocity in the distal direction and uses this velocity to strike the probe head via its impact pin, thereby transferring all the residual energy and momentum to the probe in order to shatter a calculus. Meanwhile, the acceleration tube continues to move and the distal opening remains closed and is then pushed into the overpressure region, while the previously open proximal opening is closed and then moved into the ambient pressure region. As a result, compressed air flows through the distal opening into the cavity of the acceleration tube and the projectile is accelerated in the proximal direction.

This section-by-section design of the billiard projectile in the axial direction offers the advantage that there is variance in and independence of all component dimensions, in particular of the spring element. In this way, not only the first spring element but also the second spring element can be selected and adjusted according to the intended energy flow and do not necessarily have to fit into the acceleration tube of the projectile. In this way, the required spring hardness, in particular of the distal spring element, can be realized in a dimension around the impact pin for which there is otherwise no installation space.

While at the proximal end the energy absorbed by the spring is used to move the acceleration tube further in the proximal direction and to repulse the projectile back in the distal direction, the energy transferred to the second spring element at the distal end is used to move the acceleration tube in the distal direction, to shock-excite the probe and optionally to repulse the projectile in the proximal direction. At the same time, the billiard projectile as a distal stop element provides protection for the spring and redundancy for safety in case the lithotripsy device has not been properly assembled for an operation. It is particularly advantageous that the impact pin of the billiard projectile strikes the probe before its entire kinetic energy is absorbed in the spring. In this way, the billiard projectile with the spring element serves as a shock absorber and transmitter without the formation of an air spring; consequently no venting is necessary in this region during regular operation of the lithotripsy device.

In a further embodiment of the lithotripsy device, the first spring element and at least partially the proximal stop element are accommodated in a proximal holding unit and the second spring element and at least partially the distal stop element are accommodated in a distal holding unit, wherein the proximal holding unit and the distal holding unit are each connected directly or indirectly to an outer side of the acceleration tube and are movable in the axial direction.

This provides a complete assembly with an axially movable acceleration tube, which can be easily manufactured. Because the proximal holding unit is attached from the outside to the outside of the proximal end portion of the acceleration tube and the first spring element is arranged on the inside on the proximal end followed in the distal direction by the proximal stop element, and an analogous structure is formed on the distal end with the exception of a passage opening on the distal wall of the distal holding unit for the impact pin to pass through, this entire assembly can be arranged and mounted in a simple, movable manner within the guide tube and/or the terminal end caps. The respective outer sides of the proximal holding unit and the distal holding unit preferably rest directly on the inner side of the respective end caps and/or the guide tube.

A “holding unit” is in particular a component or has multiple components which receive and hold the respective spring elements and at least in some cases the respective stop elements. In particular, an outer side of the proximal or distal end portion of the acceleration tube is fixed, directly or indirectly, to the respective holding unit. The holding unit can be made in one piece or in multiple parts. For example, the holding unit can be realized in two parts by a cap with an external thread that is fixed, for example soldered, to the outside of the acceleration tube at each end, with an end piece with an internal thread being screwed on as the second part of the holding unit. The connection between the respective holding units and the acceleration tube is in particular positive and/or non-positive. In order to make this assembly lightweight, the proximal and distal holding units are made of the thinnest material possible.

Because the holding unit is fixedly connected to the outside of the acceleration tube, the spring presses directly onto the acceleration tube via the holding unit, thereby causing the axial displacement.

In order to realize a rapid switching between the valve opening positions and to provide the pressure medium within the lithotripsy device on the proximal end and the distal end, a chamber, or two or more separate chambers, for passing pressure medium to and/or from the at least one proximal opening or openings and/or the at least one distal opening or openings is/are arranged between an outer surface of the guide tube and an inner surface of the carrier unit.

Due to the at least one chamber or preferably four separate chambers evenly distributed over the cross-section of the carrier unit on the inside thereof, which are implemented along the longitudinal direction of the carrier unit and thus of the acceleration tube, compressed air can preferably be supplied directly to both the proximal inlet air channel and the distal inlet air channel, so that a pressure medium is immediately present in both inlet air channels, regardless of the respective valve opening positions. This enables rapid switching between the first valve opening position and the second valve opening position. In contrast, the exhaust air is preferably not discharged via chambers, but, as described above, vented radially outwards to outside the lithotripsy device. The chambers can in particular be designed as longitudinal holes in the carrier unit.

In a further embodiment, the lithotripsy device has a connection port for connecting to the drive device and for continuously supplying or removing the pressure medium.

Preferably, the lithotripsy device has only a single connection port, such that the drive device can be connected to this connection port with a single hose. This makes handling of the lithotripsy device easier.

A “connection port” is any connecting element that provides a connection for the pressure medium between the drive device and the lithotripsy device. A connection port is in particular a short length of pipe, such as a hose connector, a hose nozzle or a hose coupling. A connection port can also simply be an opening in the housing wall and/or the carrier unit of the lithotripsy device. This opening can, for example, have an internal thread for screwing in a hose nozzle. Such an opening can also be designed without a thread and the pressure medium simply flows into the lithotripter through this opening.

In order to provide a comprehensive and/or autonomously operating lithotripsy device, it comprises the probe and/or the drive device.

In order to achieve the acceleration of the projectile either by means of an overpressure or a negative pressure, a negative pressure and/or an overpressure can be applied at the cavity or a part of the cavity of the acceleration tube by means of the drive device.

While overpressure requires a compressor or a building pressure line with a maximum specified pressure, operating the lithotripsy device with a negative pressure and thus creating a vacuum further reduces patient risk, simplifies the design of the corresponding control device and thus reduces costs, since complex compression, pressure control and/or pressure relief valves in the control device can be dispensed with. For example, for operating with a negative pressure, the lithotripsy device can be connected directly to an existing building vacuum supply in a clinic.

In a negative pressure operation, for example, to move the projectile to the distal stop element, instead of supplying compressed air through the proximal opening of the acceleration tube, a negative pressure is applied to the distal opening and thus the air is sucked out of the cavity of the control sleeve, thereby moving the projectile to the distal stop element. Accordingly, the processes described in this application with regard to the inflow and feed as well as the outflow and discharge of the pressure medium apply analogously to an overpressure operation, and vice versa in the case of a negative pressure operation.

In a further aspect of the invention, the object is achieved by a method for accelerating a projectile of a lithotripsy device, wherein the lithotripsy device has a guide tube and an acceleration tube with a cavity, and the acceleration tube is at least partially surrounded by the guide tube, wherein a projectile that can be moved between a resilient proximal stop element and a resilient distal stop element is arranged in the cavity of the acceleration tube, and the acceleration tube has at least one proximal opening and at least one distal opening for the inflow and/or outflow of a pressure medium into and/or out of its cavity for moving the projectile back and forth between the resilient proximal stop element and the resilient distal stop element, and a drive device for supplying and/or discharging the pressure medium, and a probe can be assigned to the lithotripsy device, and the acceleration tube is supported internally at its proximal end portion by means of the resilient proximal stop element and is arranged at its distal end portion to be movable in the axial direction by means of the resilient distal stop element, said method having the following steps:
supplying and/or discharging the pressure medium by means of the drive device and flowing the pressure medium through the at least one proximal opening into the cavity of the acceleration tube, and moving the projectile by means of the pressure medium to the resilient distal stop element,
accelerating the projectile by means of the pressure medium,
impacting the projectile on the resilient distal stop element and displacing the acceleration tube by means of the resilient distal stop element in a distal direction in order to switch the flow of the pressure medium,
optionally repulsing the projectile at the resilient distal stop element,
transferring an impact pulse of the projectile upon impact by means of the resilient distal stop element to a probe, and/or
supplying and/or discharging the pressure medium by means of the drive device and flowing the pressure medium through the at least one distal opening into the cavity of the acceleration tube, and moving the projectile back to the resilient proximal stop element by means of the pressure medium,
accelerating the projectile by means of the pressure medium,
impacting the projectile on the resilient proximal stop element and displacing the acceleration tube by means of the resilient proximal stop element in a proximal direction in order to switch the flow of the pressure medium, and
repulsing the projectile at the resilient proximal stop element.

In this way, the user can use the method very easily and quickly after starting the lithotripsy device, because of the self-controlling, axially movable acceleration tube, based on defined valve positions and switching the direction of movement to repeatedly move the projectile back and forth along the acceleration path, without having to pay attention to pressures and valve switching of an external pressure medium feed. The method described above refers to an overpressure operation, and applies analogously to a negative pressure operation, in which a suction pressure is applied to the distal opening of the acceleration tube in order to move the projectile towards the distal stop element. In the negative pressure mode, the opposite procedure is followed when the projectile is moved back to the proximal stop element and the suction pressure is applied to the proximal opening of the acceleration tube.

During the distal switching process, the kinetic energy of the projectile when it strikes the resilient distal stop element is used to move the acceleration tube further axially in the distal direction by means of force transmission, and thereby cause an automatic valve switching for the pressure medium, and at the same time to transfer part of the impulse and the residual energy to the probe by impacting the distal stop element onto the proximal end of the probe.

In a further embodiment of the method, the supply and/or discharge of the pressure medium is carried out continuously.

This allows the user to use a self-controlled method continuously in order to accelerate a projectile without having to constantly pay attention to the timing of the pressure pulse, as is the case with conventional pneumatic lithotripters.

The invention is explained in more detail below with reference to exemplary embodiments. The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the drawings:

FIG. 1 is a schematic, partially three-dimensional representation of a lithotripsy device with an axially movable acceleration tube in a starting position for accelerating a projectile in the distal direction;

FIG. 2 is a schematic sectional view of the lithotripsy device in a state when the projectile impacts a distal billiard projectile;

FIG. 3 is a schematic sectional view of the lithotripsy device during a distal switching process;

FIG. 4 is a schematic sectional view of the lithotripsy device at the beginning of an acceleration of the projectile in the proximal direction;

FIG. 5 is a schematic sectional view of the lithotripsy device when the projectile impacts the proximal stop element; and

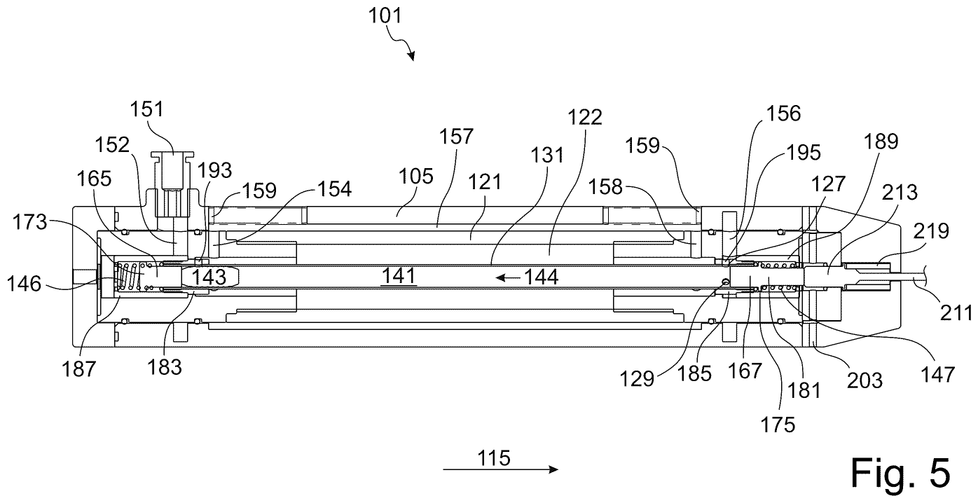

FIG. 6 is a schematic sectional view of the lithotripsy device during a proximal switching process.

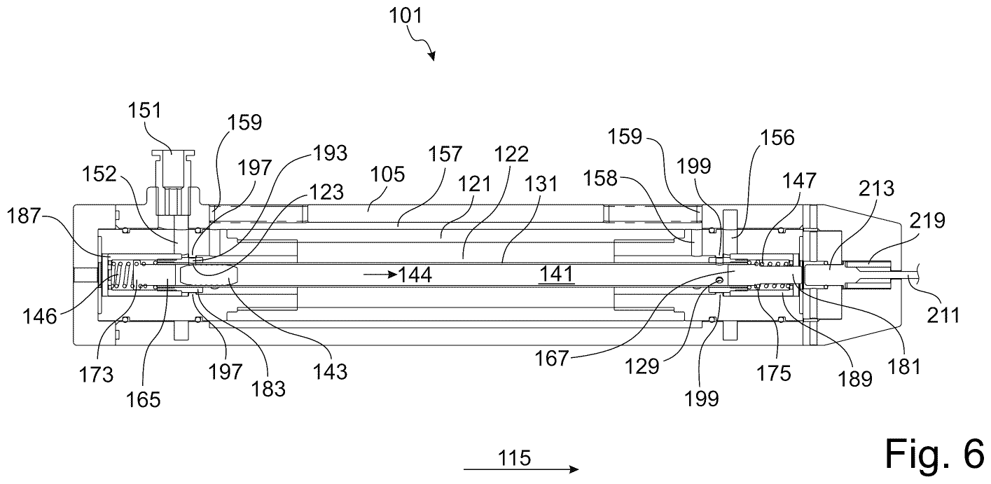

### DESCRIPTION OF PREFERRED EMBODIMENTS

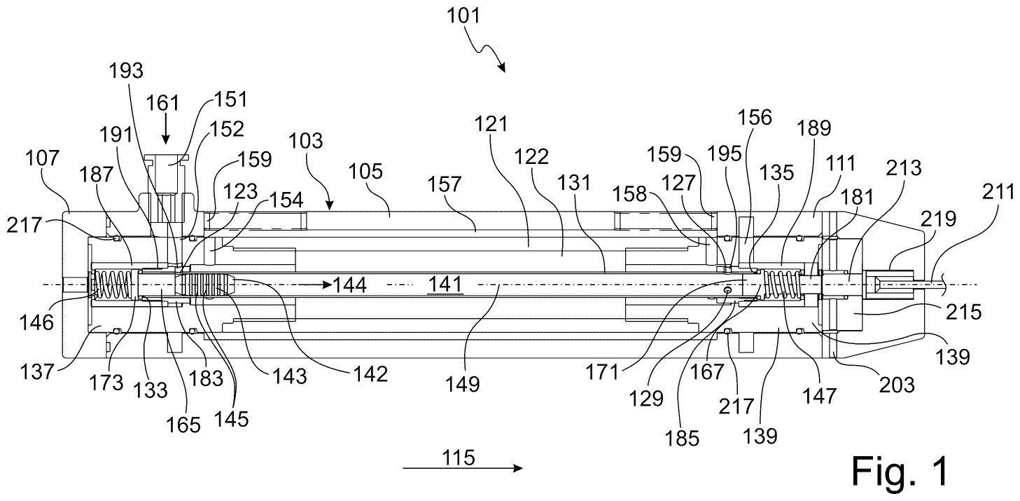

Referring to the drawings, a lithotripsy device 101 comprises a carrier unit 103 with a central housing tube 105. A proximal end of the housing tube 105 is connected to a proximal housing cap 107 and a distal end of the housing tube 105 is connected to a distal housing end cap 111 (see FIG. 1). A guide tube 121 is arranged inside the housing tube 105 of the carrier unit 103, and is connected at its proximal end by means of a proximal end cap 137 and at its distal end by means of a distal end cap 139. The proximal end cap 137 is fixed in the proximal housing cap 107 and the distal end cap 139 is fixed in the distal housing cap 111, and these are sealed respectively at the proximal end and the distal end with an O-ring 217. Between an inner wall of the housing tube 105 and an outer wall of the guide tube 121, four inlet air chambers with a symmetrical cross-section are arranged. These inlet air chambers each connect a proximal inlet air channel 152 and a distal inlet air channel 156, which are radially oriented in the respective end cap 137, 139. The inlet air chambers (not visible in FIG. 1) are fluidically connected to a compressed air connection 151 on the proximal housing cap 107, wherein inlet air is continuously supplied via the compressed air connection 151 from an external drive device (not shown).

The guide tube 121 has a cavity 122 in which an acceleration tube 131 is arranged with a longitudinal center axis 149 which runs parallel to a distal direction 115. The acceleration tube 131 has, before its proximal end 133 in a proximal end portion, a proximal opening 123 and a proximal opening 124, and two further proximal openings not visible in the figures. Likewise, the acceleration tube 131 has a distal opening 127 and a distal opening 129 before its distal end 135, as well as two further distal openings not visible in the figures. In its interior, the acceleration tube 131 has a cavity 141 which forms an acceleration path for a projectile 143 between a proximal stop element 165 and a billiard projectile 167 as a distal stop element. The proximal stop element 165 has a proximal cylinder portion 169 which is movably accommodated within the cavity 141 of the acceleration tube 131. Proximal in relation to the proximal cylinder portion 169, the proximal stop element 165 has a proximal end portion 173 which has a larger diameter than the proximal cylinder portion 169. A proximal cap 183 is soldered externally onto the proximal end portion 187 of the acceleration tube 131, and has an external thread 191 onto which a proximal end piece 187 is screwed. A proximal spring 146 is arranged between the proximal inner side of the proximal cap 183 and the proximal end face of the proximal end portion 173. The accordingly connected proximal end assembly surrounded by the proximal cap 183 is movably arranged within the cavity of the proximal end cap 137. The proximal cap 183 has a proximal through-hole 193.

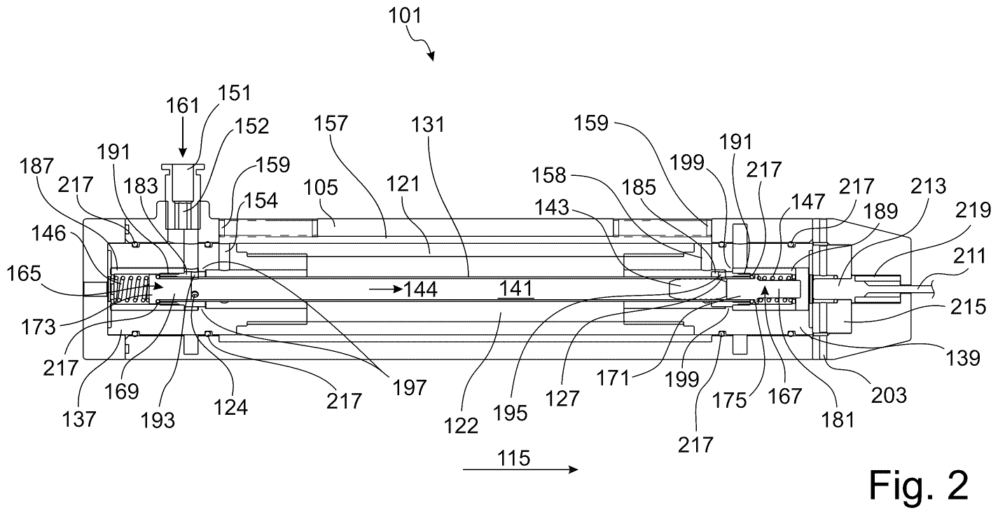

The billiard projectile 167 is arranged at the distal end portion of the acceleration tube 131. The billiard projectile 167 has a distal cylinder portion 171 which is movably arranged in the cavity 141 of the acceleration tube 131. Distal in relation to the distal cylinder portion 171, the billiard projectile 167 has a distal end portion 175, which is formed as a shoulder. Distal in relation to the distal end portion 175, the billiard projectile 167 merges into an impact pin 181, wherein the impact pin 181 has a smaller diameter than the distal cylinder portion 171. The distal end portion of the acceleration tube 131 is connected analogously to that described above by means of a soldered distal cap 185 which has an external thread 191 onto which a distal end piece 189 is screwed. The distal cap 185 has a distal through-hole 195. An O-ring 217 is arranged between the distal end 135 of the acceleration tube 131 and the proximal end of the distal end portion 175. Likewise, an O-ring 217 is arranged between the distal end of the proximal end portion 173 and the proximal end 133 of the acceleration tube 131 (see FIG. 2). In principle, it should be noted that all figures show the same lithotripsy device 101 in different states, but for reasons of clarity not all identical components in each figure are designated with the corresponding reference numerals.

The impact pin 181 formed on the distal end of the distal end portion 175 is surrounded all around by a distal spring 147 on its outer surface. The proximal spring 146 and the distal spring 147 are designed as spiral springs. Distal in relation to the distal end cap 139 is a head piece 215 in which a probe head 213 connected to an elongated probe 211 is arranged. The front part of the probe head 213 and the proximal end of the probe 211 are surrounded by a silicone tube as a damping element 219, which is supported in the distal direction 115 on the inside of the distal housing cap 111. The space around the probe head 213 is connected to the external environment of the lithotripsy device 101 via a relief hole 203 leading through the head piece 215. The probe 211 is designed as a hollow probe for comminuting calculi.

The projectile 143 is movably arranged within the cavity 141 of the acceleration tube 131. The projectile 143 has a chamfer 142 at its proximal end and at its distal end for improved movement initiation, and circumferential grooves 145 for minimizing contact.

The following work steps are carried out using the lithotripsy device 101 and the axially movable acceleration tube 131:

The lithotripsy device 101 is started by means of a control element (not shown) on the carrier unit 103, and compressed air is continuously supplied through the compressed air connection 151 in an inlet air direction 161 to the four inlet air chambers (not shown) routed in the longitudinal direction. Starting from an initial position shown in FIG. 1 for accelerating the projectile 143 in the distal direction 115, in which the proximal end piece 187 abuts at the proximal end the inner wall of the proximal housing cap 107, and thus the proximal inlet air channel 152 connected to the air chamber (not shown) is continuous with the proximal opening 123 of the acceleration tube 131 via the proximal through-hole 193 of the proximal cap 183, compressed air enters the cavity 141 of the acceleration tube 131 and presses against the projectile 143 in a projectile movement direction 144 which corresponds to the distal direction 115. At the distal end, the distal inlet air channel 156 is closed by the distal end piece 189, wherein the proximal end of the distal end piece 189 abuts a stop 199 of the distal end cap 139. The compressed air exits in the distal direction 115 from the cavity 141 through the distal openings 127, 129 into the cavity 122 of the guide tube 121 and onward through the distal outlet air channel 158. The distal outlet air channel 158 ends, just like a proximal outlet air channel 154, in a ventilation mixing chamber 157, from which the escaping air is released into the environment around the lithotripsy device 101 at the proximal end and distal end by means of ventilation channels 159. In this way, the distal openings 123, 124 are in an overpressure region, while the cavity 122 of the guide tube 121 and the ventilation mixing chamber 157, as well as the outlet air channels 154, 158 are under ambient pressure.

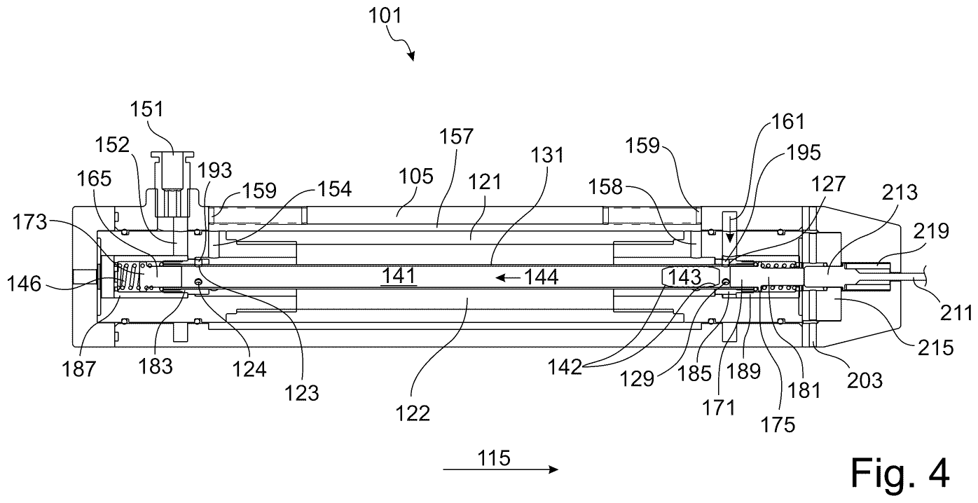

The projectile 143 is further accelerated in the same projectile movement direction 144 by the compressed air flowing in the distal direction 115 until it collides with the billiard projectile 167 and thereby transfers its momentum and kinetic energy to the billiard projectile 167 (FIG. 2). The impacted billiard projectile 167 then compresses the distal spring 147, such that, due to its spring force, the latter displaces the acceleration tube 131 further in the distal direction 115 via the connected distal cap 185 and the distal end piece 189. In this way, energy is transferred from the distal spring 147 to the acceleration tube 131. The residual velocity of the billiard projectile 167 causes the billiard projectile 167 to continue moving in the distal direction 115 and the impact pin 181 to impact the probe head 213, such that the remaining residual energy and momentum are transferred to the probe 211 in order to excite vibration of the probe 211. This transferred deformation energy can be used to comminute a calculus. In this way, the distal end piece 189 rests against the proximal end of the distal housing cap 111 in a distal starting position (FIG. 4).

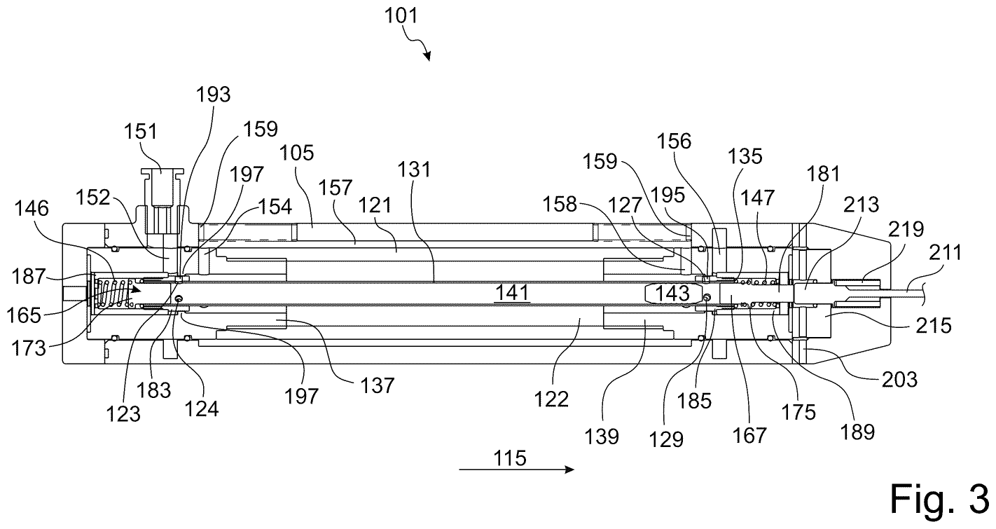

At the same time, the acceleration tube 131 moves further in the distal direction 115, such that the proximal end piece 187 increasingly closes the proximal inlet air channel 152 until the distal end of the proximal end piece 187 abuts a stop 197 of the proximal end cap 137. At the same time, the proximal openings 123, 124 are displaced in the distal direction 115, closed by the externally adjacent proximal end cap 137 (see FIG. 3) and then moved further into the surrounding area. Meanwhile, the closed distal openings 127, 129 are moved further in the distal direction 115 until they, together with the distal through-hole 195, are continuous with the distal inlet air channel 156 and are then in the overpressure region. The projectile 143 actively repulsed at the billiard projectile 167 now moves in the projectile movement direction 144 in the proximal direction opposite the distal direction 115, so that an automatic switching of the direction of movement has taken place. The projectile is further accelerated in the proximal direction by the compressed air flowing in via the chambers (not shown) through the distal inlet air channel 156, the distal through-hole 195 and the distal openings 127, 129 until it impacts the proximal stop element 165 at the proximal end and transfers its entire momentum to the proximal stop element 165 due to the equal mass ratios between the projectile 143 and the proximal stop element 165. Via the proximal spring 146, a force is transmitted to the acceleration tube 131 in the same way as described for the distal spring 147, thereby displacing the acceleration tube 131 further in the proximal direction until the proximal end piece 187 again on the inside abuts the distal end of the proximal housing cap 107 (FIG. 1).

These processes described above of accelerating the projectile 143 and axially displacing the acceleration tube 131 in the distal direction 115 and in the opposite direction in the proximal direction are repeated automatically without any further user intervention. In this way, a lithotripsy device 101 is provided in which an automatic valve changeover for moving a projectile 143 back and forth is realized by means of the axially movable acceleration tube 131, the proximal stop element 165 and the billiard projectile 167, wherein the processes are automatically repeated in a clocked manner with a continuous flow through the acceleration tube 131 itself. This eliminates the need for complex control and valve switching of an external, intermittent compressed air supply.

While specific embodiments of the invention have been shown and described in detail to illustrate the application of the principles of the invention, it will be understood that the invention may be embodied otherwise without departing from such principles.

### LIST OF REFERENCE SIGNS

101 Lithotripsy device
103 Carrier unit
105 Housing tube
107 Proximal housing cap
111 Distal housing cap
115 Distal direction
121 Guide tube
122 Cavity of the guide tube
123 Proximal opening
124 Proximal opening
127 Distal opening
129 Distal opening
131 Acceleration tube
133 Proximal end of the acceleration tube
135 Distal end of the acceleration tube
137 Proximal end cap
139 Distal end cap
141 Cavity/acceleration path
142 Chamfer
143 Projectile
144 Projectile movement direction
145 Grooves
146 Proximal spring
147 Distal spring
149 Longitudinal center axis
151 Compressed air connection
152 Proximal inlet air channel
154 Proximal outlet air channel
156 Distal inlet air channel
157 Ventilation mixing chamber
158 Distal outlet air channel
159 Ventilation channel
161 Inlet air direction
165 Proximal stop element
167 Billiard projectile (distal stop element)
169 Proximal cylinder portion
171 Distal cylinder portion
173 Proximal closure portion
175 Distal closure portion
181 Impact pin
183 Proximal cap
185 Distal cap
187 Proximal end piece
189 Distal end piece
191 External thread
193 Proximal through-hole
195 Distal through-hole
197 Proximal end cap stop
199 Distal end cap stop
203 Ventilation hole
211 Probe
213 Probe head
215 Head piece
217 O-ring
219 Damping element

1. A lithotripsy device for breaking up calculi, wherein the lithotripsy device comprises
a carrier unit,
a guide tube,
an acceleration tube with an axial direction, a cavity, a proximal end and a distal end, a movable projectile, and a proximal stop element and a distal stop element for the movable projectile, wherein the acceleration tube is at least partially surrounded by the guide tube and the acceleration tube has at least one proximal opening and at least one distal opening for the inflow and/or outflow of a pressure medium into and/or out of its cavity, for the back and forth movement of the projectile between the proximal stop element and the distal stop element, and the lithotripsy device can is configured to be assigned a drive device for supplying and/or discharging the pressure medium, and is configured to be assigned a probe, wherein the probe can be connected directly or indirectly to the carrier unit at a carrier unit proximal end and can be excited to vibrate by a mechanical impact of the projectile on the distal stop element, wherein the acceleration tube is arranged to be movable in the axial direction internally at an acceleration tube proximal end portion by means of the proximal stop element and at an acceleration tube distal end portion by means of the distal stop element, so that the acceleration tube is displaceable in a distal direction and in a proximal direction relative to the guide tube.

2. A lithotripsy device according to claim 1, wherein for a proximal stop of the axially movable acceleration tube, a first valve opening position is created for the pressure medium to flow through the at least one proximal opening into and/or out of the cavity of the acceleration tube, for moving the projectile towards the distal stop element, or for moving the projectile back towards the proximal stop element, and for a distal stop of the axially movable acceleration tube, a second valve opening position is created for the pressure medium to flow through the at least one distal opening into and/or out of the cavity of the acceleration tube, for moving the projectile back towards the proximal stop element or for moving the projectile to the distal stop element.

3. A lithotripsy device according to claim 1, wherein the acceleration tube has a second proximal opening and/or further proximal openings and a second distal opening and/or further distal through-openings.

4. A lithotripsy device according to claim 1, wherein the at least one proximal opening or openings and the distal opening or openings are arranged in a peripheral surface of the acceleration tube, axially symmetrical to a longitudinal center axis of the acceleration tube and/or all around the circumference.

5. A lithotripsy device according to claim 1, wherein the proximal stop element has a first cylindrical portion and the distal stop element has a second cylindrical portion, wherein the proximal end portion of the acceleration tube is arranged to be movable in the axial direction around the first cylindrical portion and the distal end portion of the acceleration tube is arranged to be movable around the second cylindrical portion.

6. A lithotripsy device according to claim 1, wherein the proximal stop element has a first end portion as a stop on the proximal end of the acceleration tube and the distal stop element has a second end portion as a stop on the distal end of the acceleration tube.

7. A lithotripsy device according to claim 1, wherein a first spring element for repulsing the projectile is arranged proximally in relation to the proximal stop element and/or proximally in relation to the first closure element.

8. A lithotripsy device according to claim 5, wherein the distal stop element has an impact pin distally in relation to the second cylindrical portion and/or to the second end portion for transmitting an impact of the projectile to the probe.

9. A lithotripsy device according to claim 7, wherein the distal stop element has an impact pin distally in relation to the second cylindrical portion and/or to the second end portion for transmitting an impact of the projectile and wherein a second spring element is arranged distally in relation to the second end portion and/or around the impact pin of the distal stop element.

10. A lithotripsy device according to claim 9, wherein the first spring element and at least partially the proximal stop element are accommodated in a proximal holding unit and the second spring element and at least partially the distal stop element are accommodated in a distal holding unit, wherein the proximal holding unit and the distal holding unit are each directly or indirectly connected to an outer side of the acceleration tube and are movable in the axial direction.

11. A lithotripsy device according to claim 1, wherein a chamber, or two or more separate chambers, for conveying pressure medium to and/or from the at least one proximal opening or the proximal openings and/or the at least one distal opening or the distal openings is/are arranged between an outer surface of the guide tube and an inner surface of the carrier unit.

12. A lithotripsy device according to claim 1, wherein the lithotripsy device has a connection port for connecting to the drive device and for continuously supplying or discharging the pressure medium.

13. A lithotripsy device according to claim 1, wherein, by means of the drive device, a negative pressure and/or an overpressure can be applied to the cavity or a part of the cavity of the acceleration tube.

14. A method for accelerating a projectile of a lithotripsy device, wherein the lithotripsy device comprises a guide tube and an acceleration tube with a cavity, and the acceleration tube is at least partially surrounded by the guide tube, wherein a projectile movable between a resilient proximal stop element and a resilient distal stop element is arranged in the cavity of the acceleration tube, and the acceleration tube has at least one proximal opening and at least one distal opening for the inflow and/or outflow of a pressure medium into and/or out of its cavity for the back and forth movement of the projectile between the resilient proximal stop element and the resilient distal stop element, and the lithotripsy device is configured to be assigned a drive device for supplying and/or discharging the pressure medium and a probe, and the acceleration tube is arranged to be movable in the axial direction on the inside at an acceleration tube proximal end portion by means of the resilient proximal stop element and at an acceleration tube distal end portion by means of the resilient distal stop element, having the following steps:
supplying and/or discharging the pressure medium by means of the drive device and flowing the pressure medium through the at least one proximal opening into the cavity of the acceleration tube, and moving the projectile by means of the pressure medium to the resilient distal stop element,
accelerating the projectile by means of the pressure medium,
impacting the projectile on the resilient distal stop element and moving the acceleration tube by means of the resilient distal stop element in a distal direction to switch the flow of the pressure medium,
transmitting an impact pulse of the projectile upon impact by means of the resilient distal stop element to a probe, and/or
supplying and/or discharging the pressure medium by means of the drive device and flowing the pressure medium through the at least one distal opening into the cavity of the acceleration tube, and moving the projectile back by means of the pressure medium to the resilient proximal stop element,
accelerating the projectile by means of the pressure medium,
impacting the projectile on the resilient proximal stop element and displacing the acceleration tube by means of the resilient proximal stop element in a proximal direction to switch the flow of the pressure medium, and
repulsing the projectile on the resilient proximal stop element.

15. A method according to claim 14, wherein the supply and/or discharge of the pressure medium is carried out continuously.
